(19) United States
(12) Patent Application Publication
(10) Pub. No.: US 2013/0211265 A1
(43) Pub. Date: Aug. 15, 2013

(54) MULTIFUNCTIONAL MEDICAL DEVICE FOR TELEMEDICINE APPLICATIONS

(71) Applicants: William Bedingham, Woodbury, MN (US); Thomas P. Schmidt, Blaine, MN (US); Bernard A. Gonzalez, St. Paul, MN (US); Shaun M. Krueger, South St. Paul, MN (US)

(72) Inventors: William Bedingham, Woodbury, MN (US); Thomas P. Schmidt, Blaine, MN (US); Bernard A. Gonzalez, St. Paul, MN (US); Shaun M. Krueger, South St. Paul, MN (US)

(73) Assignee: 3M INNOVATIVE PROPERTIES COMPANY, St. Paul, MN (US)

(21) Appl. No.: 13/879,643

(22) PCT Filed: Oct. 12, 2011
(86) PCT No.: PCT/US2011/055867

§ 371 Date: Apr. 16, 2013

Related U.S. Application Data
(60) Provisional application No. 61/394,076, filed on Oct. 18, 2010.

(51) Int. Cl. A61B 5/00

(52) U.S. Cl. CPC A61B 5/0024
USPC 600/483; 600/300

(57) ABSTRACT

A multifunction medical device includes a housing configured for hand-held manipulation, a processing unit within the housing, and a connector on the outer surface of the housing. The connector is coupled to the processing unit and operable to connect to a plurality of different types of medical sensors. The processing unit is configured to determine a type of a medical sensor coupled to the connector.

### CROSS REFERENCE TO RELATED APPLICATIONS

This application claims priority to U.S. Provisional Patent Application No. 61/394,076 and U.S. Provisional Patent Application No. 61/394,083, both filed on Oct. 18, 2010, which are incorporated herein by reference.

### BACKGROUND

Medical devices are used in measuring vital signs of human body and/or examining the human body. For example, a stethoscope is typically used to detect acoustic signals representative of cardiovascular or pulmonary function generated by a body. A pulse oximeter provides an indirect measurement of the oxygen saturation of a patient's blood and often measures the heart rate of the patient. An otoscope is used to look into a patient's ear canals to screen for illness. An electrocardiography (ECG) monitors the electrical activity of the heart over time. An endoscope is an instrument used to examine the interior of a hollow organ or cavity of the body.

Telemedicine is designed to provide medical diagnosis and treatment to patients remotely. It allows patients access to healthcare in underserved areas, such as rural communities. It also reduces cost and resource consumption for providing health care. A variety of medical devices have been developed to use for telemedicine applications. In telemedicine applications, medical information is collected from medical devices and transferred through media communication network for the purpose of remote consultations, medical examinations, or medical procedures.

### SUMMARY

In one embodiment, a multifunctional medical device, consistent with the present invention, comprises a housing configured for hand-held manipulation, a processing unit within the housing, a first medical sensor coupled to the processing unit for generating medical measurement signals, and a connector. Additionally, the connector is coupled to the processing unit and operable to connect to a plurality of different types of medical sensors, each type of medical sensors operable to generate corresponding medical measurement signals. The processing unit is configured to determine a type of a second medical sensor connected to the connector, receive medical measurement signals from the first medical sensor and the second medical sensor connected to the connector, and transmit the received signals via a network. The first medical sensor and the second medical sensor operate simultaneously when in use.

In another embodiment, a multifunctional medical device comprises a housing configured for hand-held manipulation, a processing unit within the housing, an exam camera coupled to the processing unit, and a connector. The housing has a first end and a second end opposite the first end. The exam camera is positioned on the first end of the housing. The connector is on the outer surface of the housing. Additionally, the connector is coupled to the processing unit and operable to connect to a plurality of different types of medical sensors, each type of medical sensors operable to generate corresponding medical measurement signals. The processing unit is configured to determine a type of a medical sensor connected to the connector, receive medical measurement signals from the exam camera and the medical sensor connected to the connector, and transmit the received signals via a network.

### BRIEF DESCRIPTION OF THE DRAWINGS

The accompanying drawings are incorporated in and constitute a part of this specification and, together with the description, explain the advantages and principles of the invention. In the drawings,

FIG. 1a illustrates an embodiment of a multifunctional medical device;

FIG. 1b is a perspective view of an embodiment of a multifunctional medical device;

FIG. 2 illustrates an embodiment of a telemedicine companion system;

FIG. 3 is a block diagram of an exemplary multifunctional medical device;

FIG. 4 is an exemplary functional module for a processing unit;

FIG. 5 is a system diagram of an exemplary multifunctional medical device;

FIG. 6 and FIG. 7 are exemplary flowcharts for a medical sensor discovery process of a multifunctional medical device;

FIG. 8 is a system diagram of an exemplary telemedicine companion system;

FIG. 9 is an illustration of an exemplary telemedicine companion system;

FIG. 10 and FIG. 11 are exemplary functional flowcharts of a telemedicine companion system;

FIG. 12 is a flowchart of an exemplary device registration procedure of a telemedicine companion system;

FIG. 13a and FIG. 13b is an exemplary flowchart for a medical sensor discovery process of a telemedicine companion system;

FIG. 14 is a screen shot of an embodiment of a user interface on a companion device;

FIG. 15 is a screen shot of an embodiment of a user interface on a companion device; and

FIG. 16 is another screen shot of an embodiment of a user interface on a companion device.

### DETAILED DESCRIPTION

Telemedicine allows a medical specialist to provide medical diagnosis and treatment to a patient at a second, often remote location. Typically, the patient is assisted with a nurse or other care provider, but may interact with the specialist without local assistance. It would be highly desirable in such situations to provide a portable medical device at the patient site that can perform adequate medical examinations of the patient and provide accurate medical measurement data to the specialist site in a secured and timely manner. The present disclosure is directed to a multifunctional medical device that collects data from one or more medical sensors permanently or temporarily associated therewith, transmits the data to the specialist site by a secured network and includes an open architecture allowing connection of various medical sensors.

As used herein, a medical sensor is a device that is used in medical examinations, medical diagnosis, or medical procedures. For example, a medical sensor may be an acoustic biosensor, a set of electrocardiography electrodes, a glucose meter, a pulse oximeter, an exam camera, a sphygmomanometer, a spirometer, a thermometer, an endoscope, an ophthalmoscope, an otoscope, and the like. Medical measurement signals refer to analog or digital signals collected or generated by one or more medical sensors. Such signals may or may not undergo signal processing.

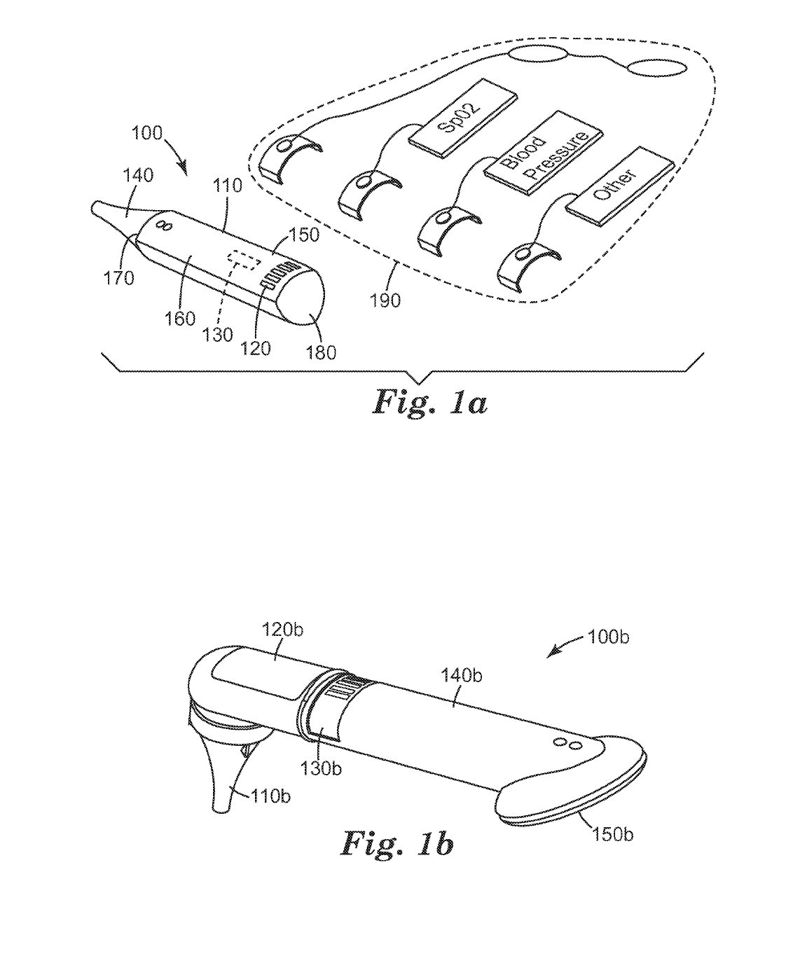

FIG. 1a illustrates an embodiment of a multifunctional medical device 100. Device 100 includes a housing 110, a connector 120, and a processing unit 130 disposed at least partially within the housing. As used herein, a connector refers to a sensor interface that is operable to provide connection to different types of sensors, including but not limited to, medical sensors. The device 100 may have an optional medical sensor 140 coupled to the processing unit 130.

In certain embodiments, the housing 110 is designed to interface with human body, which means that the device may be in direct contact or in close proximity with human body. In some cases, the housing 110 may have an outer surface 150, an elongated body 160, a first end 170, and a second end 180. The elongated body 160 provides easy hand-held manipulation. In some cases, the elongated body 160 is essentially cylindrical, though additional shapes are contemplated, including but not limited to those with a profile graspable by hand. The housing 110 may be unitary and/or hermetically sealed. The housing may be implemented with a material capable of being sealed and easy to clean, such as metal or plastic. In some embodiments, the housing 110 is water-proof.

The connector 120 may be on the outer surface of the housing. In some cases, the connector 120 may be flush with the outer surface of the housing such that the device 100 is easy to clean and less susceptible to microbial growth. In such a configuration, the connector 120 is not substantially protruding or indented from the outer surface of the housing. Medical sensor(s) 190 illustrate an example connector that may be used to establish connection to the connector 120. Once the connector 120 is connected with a medical sensor, the processing unit 130 is configured to automatically recognize the type of the medical sensor. In some cases, the processing unit may download a driver applicable to the type of the medical sensor after the type is recognized.

In an exemplary embodiment, the medical sensor 140 may be positioned at the first end 170 or the second end 180 of the housing 110. In a particular embodiment, the medical sensor 140 is a medical exam camera positioned on the first or the second end of the housing 110. As used herein, an exam camera, or referred to as a medical exam camera, is a visualization and/or imaging device that can be used in medical examination and testing environment, for example, an endoscope, an otoscope, a medical infrared camera, or the like. In another embodiment, the medical sensor 140 comprises a transducer configured to detect auscultation sounds.

In a preferred embodiment, a medical device 100b includes a medical exam camera 110b proximate the first end of the housing and a transducer detecting auscultation sounds 150b proximate the second end of the housing, as illustrated in FIG. 1b. The medical device 110b has a housing 140b and a connector 130b. In some embodiments, the medical device 100b includes a user interface 120b. With an elongated body, the device may be designed to allow the user interface 120b to be at least partially visible and accessible, while the device is in hand-held use. When the device 100b is in use for examination using camera 110b, a user would typically grip the device around the housing section 140b and point the camera 110b at the patient. In such a configuration when the device is in use, the user interface 120b is facing and thus visible to the user, allowing the user to view information on the user interface or enter commands via the user interface when implemented with a touch screen display. Also, buttons or switches for entering control information are typically positioned on the same side of the housing as the user interface so that they are facing the user and easily accessible when the device is in use.

The multifunctional medical device 100 has features suitable for telemedicine applications. First, the connector 120 allows the multifunctional medical device 100 to be easily expanded to perform various types of medical examinations. Second, the automatic recognition of the type of medical sensor connected to the connector 120 allows the device to be used with minimum training Third, the small form factor of the device 100 is portable and comfortable to use. In particular, the device preferably has a form factor small enough to allow it to be easily used by hand. Fourth, the device 100 may have a flush surface that is easy to clean and sanitize. While the multifunctional medical device 100 is suitable for telemedicine applications, it may be used in a clinical service environment.

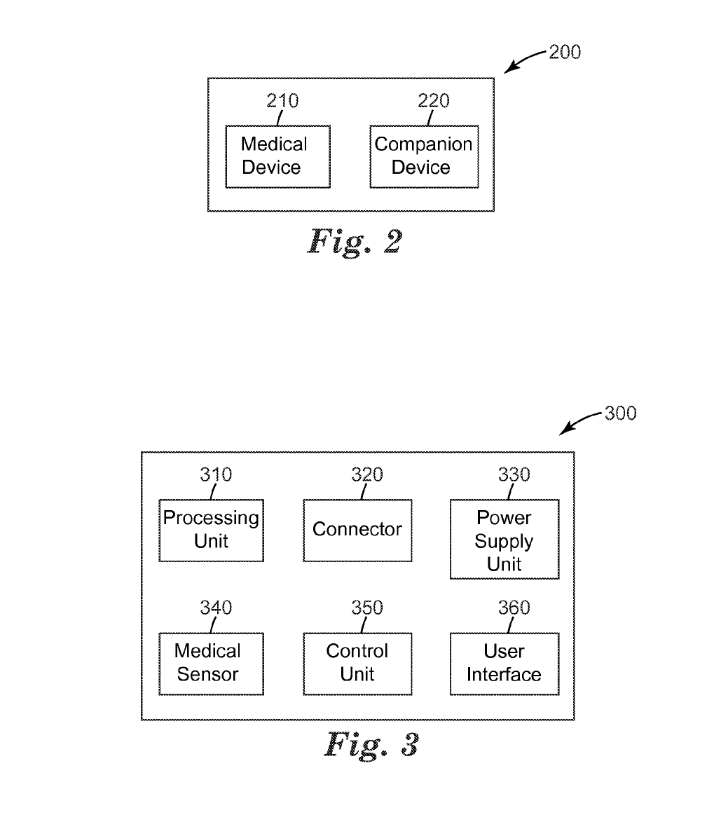

Another issue for telemedicine applications is the transmission of medical measurement data across a wide geographical area, where network connection is often limited. In many situations, the medical measurement data need to be transmitted through a network security firewall to reach the specialist site. A network firewall blocks unauthorized communication entering and leaving an intranet. The present disclosure teaches a telemedicine companion system that is capable of providing reliable transmission of medical measurement data substantially real-time. FIG. 2 illustrates an embodiment of a telemedicine companion system 200. In one embodiment, the telemedicine companion system 200 comprises a medical device 210 and a companion device 220. The medical device 210 and the companion device 220 may each be connected to secured wireless networks. In one embodiment, the medical device is a hand-held medical device with wireless capability. In some embodiments, the medical device is a multifunctional medical device described in the present disclosure.

The companion device 220 receives medical measure signals from the medical device 210 via a secured wireless network. In some cases, the companion device may provide a user interface to the medical device 210. For example, the companion device may have a display to present the medical measurement signals generated by the medical device. The companion device 220 may load a user interface unique to the type of the medical device to provide control to the medical device 210 and display data received from the medical device 210. In certain embodiments, the companion device 220 is a cellular phone. In some cases, the companion device 220 is connected with the specialist site via a secured wireless network. In some other cases, the companion device 220 may allow a care giver at the patient site to ask questions, receive instructions, and provide feedback from a medical specialist at a remote location. In some cases, the companion device 220 is used at the specialist site. In some embodiments, the medical device 210 and the companion device 220 are registered and authorized by a server before the medical device 210 is able to communicate with the companion device 220. In some other embodiments, the medical device 210 may store medical measurement data in its internal memory and transmit the data to the companion device later, which is referred to hereafter as store-and-forward mode. When the network is unavailable or has limited bandwidth, the store-and-forward mode allows robust data transmission at a later time.

In some embodiments, the companion device 220 may be a mobile device, for example, a cellular phone, a smart-phone, a personal digital assistant (PDA), a laptop, a tablet personal computer (PC), and the like. Because mobile devices are used extensively, a care giver at the patient site may use an available mobile device instead of acquiring a new one. It is often desirable to have a separate medical device 210 distinct from the companion device 220, such that the medical device 210 may be sanitized adequately prior to and after contact with a patient.

In a preferred embodiment, the medical device 210 and the companion device 220 are connected to cellular networks, preferably secured cellular networks. In some developing countries, land-line telephone infrastructures are aging and inadequate, but the newly installed cellular networks can reach a majority of rural areas and villages. Thus, the telemedicine companion system 200 provides a low-cost means to transmit reliable, high-quality, real-time medical data to a remote specialist site in those areas, although the system can also be used anywhere a telecommunication network is available.

A cellular network is a radio network distributed over land areas (i.e. cells) which each cell is served by at least one fixed location transceiver. Cellular networks encompass CDMA, GSM, 3G, 4G Networks, and any other cellular network. Cellular connection allows the medical device 210 and the companion device 220 to be untethered, where the medical device 210 and the companion device 220 do not need to be physically connected, or within a short distance as required by Bluetooth technology, or in an area covered by wireless access point for short-range wireless interface. Additionally, cellular connection provides communication across a firewall. For example, the companion device 220 may transmit medical examination data to a server within a firewall of a medical facility. Furthermore, cellular connection enables same user experience at multiple locations, including, but not limited to, home, clinic, ambulance, or hospital. Cellular connection is also capable to provide health condition of a patient collected by the medical device 210 while the patient is in-transit. Further, a secured cellular connection maintains privacy of patient data and information. In summary, the use of a cellular network provides a widely-used, robust, secure, and high bandwidth communication channel.

Multifunctional Integrated Medical Device

FIG. 3 is a block diagram of an exemplary multifunctional medical device 300. The multifunctional medical device 300 has a processing unit 310, a connector 320, and a power supply unit 330. Optionally, the multifunction medical device 300 may have a medical sensor 340, a control unit 350, and a user interface 360. The medical sensor 340 may be electronically coupled to the processing unit 310. In some embodiments, the connector 320 is configured to allow wired connection, or wireless connection, or both types of connection of various types of medical sensors. In some embodiments, the connector 320 is configured to receive analog and digital signals. Additionally, the connector 320 may be connected with peripheral devices to provide information regarding the patient's physical conditions and/or environmental data of the patient site. The peripheral devices may be, for example, a scale, a pedometer, a medication monitor, a carbon monoxide detector, a smoked detector, a fire detector, a bed occupancy sensor, and the like.

In an exemplary embodiment, the connector 320 may comprise a set of connection channels for analog signals and a set of connection channels for digital signals to provide physical connection. For example, the connector 320 may be an 8-pin connector with four analog ports and four digital ports. In another exemplary embodiment, the connector 320 may internally remap its pin configuration according to the type of the medical sensor connected. The connector 320 may also provide power to the sensor. In some cases, the connector 320 may include connection channels for power input to the device and power output to an attached sensor, sensor data input. In some cases, the connector 320 may further include connection channels for sensor identification input and/or sensor control output.

In some embodiments, the medical sensor 340 and one or more additional medical sensors connected with the connector 320 may operate simultaneously. As used herein, operating simultaneously means that two or more sensors may generate medical measurement signals at the same time, in an ordered sequential manner such as with multiplexing, with temporary overlap, or by way of sampling. In some embodiments, the connector 320 may provide wireless connection to more than one medical sensor that operates simultaneously. In some embodiments, after additional medical sensors are connected with the connector, the processing unit 310 may download a driver corresponding to the additional medical sensors via a network if the driver is needed to support the additional medical sensors.

In a preferred embodiment, the multifunctional medical device 300 is rechargeable. The power supply unit 330 may include one or more rechargeable batteries. In some cases, however, the power supply unit 330 may include one or more disposable batteries. In some cases, the connector 320 may be connected to a power source to provide charging to the power supply unit 330. In some other cases, the power supply unit 330 may have a dedicated port to connect to an external power source for charging. In certain embodiments, all associated medical sensors are rendered inoperable when a connection to an external power source is established.

In some embodiments, the multifunctional medical device 300 comprises the control unit 350 is for use in generating control information. The control unit 350 may include a number of control elements (e.g., buttons, switches) to control aspects of the device operation. For example, control elements may include volume or gain control switches, or mode selection switches. In some embodiments, the control unit 350 may include one or more sensors to detect the operational condition of the multifunctional medical device 300. For example, the control unit 350 may include at least one of an accelerometer, a gyroscope, an audio transducer, an ambient light sensor, and a touch sensor. In some cases, at least part of the control unit 350 may be disposed in the housing. In some cases, the control unit 350 may include an identification reader. For example, an identification reader may be a RFID reader, a digital identification reader, a fingerprint sensor, a barcode reader, or the like. In some cases, the control unit 350 may include any traditional input devices, such as a keyboard.

In one embodiment, the control unit 350 may include a GPS unit for determining geographically related information (e.g., location). The processing unit may receive the geographically related information from the GPS unit and transmit the geographically related information via a network. In some cases, geographically related information of the patient site may be useful in determining the closest hospital in the event of emergency medical conditions. Further, cooperation between the GPS, processing unit, and the companion device via a wireless network may provide assistance in locating a lost or misplaced multifunctional device.

The user interface 360 is another optional component of the multifunctional medical device 300. The user interface 360 may include a number of mode and/or status indicators. The indicators may provide an indication of a selected filter mode, battery status, communication link status, or other information. Such communication link status indication may be based on error detection (e.g., cyclic redundancy check (CRC)) performed by the processing unit 310. In preferred embodiments, status indicative of the occurrence of an error, and not the lack thereof, is reported.

The user interface 360 may include a display suitable for installation on a hand-held device. As an example, the display may be a LCD display. In some embodiments, the user interface includes more than one display. The user interface 360 may present the status of the device, the type(s) of the active medical sensor(s), and/or received signals on the display. In some embodiments, the user interface 360 may include a touch sensitive display. In such configurations, the control unit 350 may include the same touch sensitive display.

In a particular embodiment, the multifunctional medical device 300 has an elongated body, and the device 300 is designed such that the user interface 360 is at least partially viewable and/or the control unit 350 is at least partially accessible while the device 300 is in hand-held use. In such configurations, a user may adjust the configuration of the device 300 while the user is examining a patient.

In some embodiments, based on the input of the control unit 350 or the type of the active medical sensor coupled with the processing unit 310, the configuration of the multifunctional medical device, including but not limited to, the function of the control unit 350 and/or the configuration of the user interface 360, may be changed. Here, an active medical sensor refers to a medical sensor transmitting signals to the processing unit. As an example, when the active medical sensor is changed from an acoustic sensor to an exam camera, the switches on the device are changed from volume control to zoom control. As another example, when the orientation of the device 300 being held is changed, the orientation of the display may be changed accordingly and automatically. The orientation and other motion cues (i.e. signals from internal accelerometer) of the device 300 may indicate how the device 300 is being used. For example, the device 300 may be turned on or woken from a standby mode wake up by tapping or shaking

The user interface 360 may change according to the manner of using the device 300. In some embodiments, the user interface 360 includes a touch sensitive display, and the touch sensitive display is remapped according to the input of the control unit 350 and/or the type of active medical sensor coupled to the processing unit 310. As used herein, a remapped touch sensitive display means that the display includes, compared with its original configuration, a different set of control components (e.g. buttons, checkboxes, etc.) and presentation components (e.g. textboxes, images, etc.) and/or arrange the control and presentation components differently on the screen.

In an exemplary embodiment, the multifunctional medical device 300 can include an exam camera, such as otoscope, an endoscope, or the like, positioned at a first end of the device housing. When the multifunctional medical device 300 is held upright and the exam camera is positioned at the top, the multifunctional medical device 300 can be automatically changed to a medical imaging mode where the control unit 350 and/or the user interface 360 can be configured to operate accordingly. For example, the control unit 350 can be configured to control the zooming of the exam camera. As another example, the user interface 360 can be configured to provide proper presentation components and/or display image in a proper orientation.

In another exemplary embodiment, the multifunctional medical device 300 can include a chest piece encapsulating a transducer at a second end of the device housing. When the multifunctional medical device 300 is held upright and the chest piece is positioned at the top end of the device, the multifunctional medical device 300 can be automatically changed to a medical auscultation mode where the control unit 350 and/or the user interface 360 can be configured to operate accordingly. For example, the control unit 350 can be configured to control the amplification of the signals generated by the transducer. As another example, the user interface 360 can be configured to proper presentation components and/or display image in a proper orientation.

The processing unit 310 may comprise one or more microprocessors, digital signal processors, processors, PICs (Programmable Interface Controllers), microcontrollers, or any other form of computing unit. For example, the processing unit 310 may be implemented by a Qualcomm WMD 6055 Module, a Texas Instruments OMAP3530, a Samsung S5PC 110, or one of the Qualcomm Snapdragon Platform Chipsets. The processing unit 310 receives signals from either the coupled medical sensor 340 or one or more medical sensors connected to the connector 320. The received signals may be, for example, digital streaming signals, digital discrete signals, analog streaming signals, or analog values.

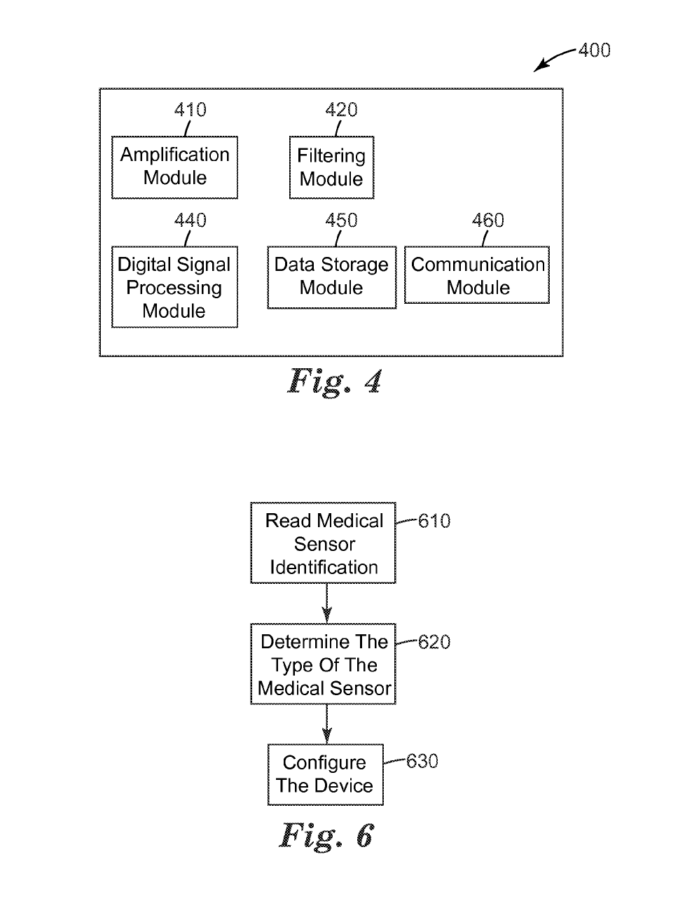

The processing unit may be configured to perform a variety of functions, ranging from simple to complex. FIG. 4 illustrates some exemplary functional modules that may be included in a processing unit 400. In some cases, the processing unit 400 may have an amplification module 410 to amplify the received signals. In some cases, the processing unit 400 may have a filtering module 420 to perform analog and/or digital filtering to enhance the quality of the received signals. In some cases, the processing unit 400 may have a digital signal processing module 440 to perform relatively sophisticated analysis on the received signals. For example, the digital signal processing module 440 may perform a profile matching with a stored profile of a patient and generate a diagnostic report.

In some cases, the processing unit 400 may convert the received signals to digital signals for accurate and faithful reproduction of medical measurement signals. For example, when an acoustic biosensor is coupled with the processing unit for collecting body sounds of a patient, the processing unit 400 may convert the received signals to acoustic signals for accurate and faithful reproduction of body sounds. In some embodiments, the processing unit 400 is configured to generate acoustic signals as described in U.S. Pat. No. 6,134,331, entitled “Electronic Stethoscope,” U.S. Pat. No. 7,006,638, entitled “Electronic Stethoscope,” and/or U.S. Pat. No. 7,130,429, entitled “Method and an Apparatus for Processing Auscultation Signals,” each of which is incorporated herein by reference in its entirety.

In some cases, when the received signals are images, the digital signal processing module 440 may perform image processing to the received signals. For example, when an exam camera is coupled with the processing unit 400, the digital signal processing unit 440 may perform pattern recognition on the received image and compress the received images to suitable size. In some cases, the digital signal processing unit 440 may packetize the received signals or the processed data generated by the other modules. In some cases, the digital signal processing unit 440 may facilitate auto focus of exam camera optics.

In some embodiments, the processing unit 400 may have a data storage module 450 to store the received signals and/or the processed data generated by other modules within the processing unit 400. In some cases, the data storage module 450 may allow a user to store voice tracks, physiological signals, configuration preference, or other data. The data storage module 450 may further include voice recognition data to identify the user or owner of the medical device and speech recognition data to identify voice commands so that certain settings of the multifunctional medical device may be modified in response to voice commands. Speech recognition voice commands may also be used to transfer voice tracks, body sounds, or other recordings or files to patient medical records. In some embodiments, the multifunctional medical device is configured to transcribe the content of voice signals into records or other data files (e.g., patient medical records), as described, for example, in U.S. Pat. No. 7,444,285 (Forbes).

In one embodiment, the processing unit 400 may include a communication module 460 to receive and transmit signals via a network. The signals transmitted from the communication module 460 may be, for example, the received signals from the active medical sensors, the processed data based on the received signals, or the data packets generated by the digital signal processing unit 440. The communication module 460 may receive commands from a companion device. As used herein, a command may be a command to change a device configuration, a command to change operation of the device, a request for data, or the like. When the network is not available, the processing unit 400 may store the data to be transmitted in the data storage module 450. The processing unit 400 may later transmit the data via the communication module 460 when the network is available. Further, the processing unit 400 may re-transmit data saved in the data storage module 450 if the data is lost during the transmission or upon request from the specialist site or from a companion device. The processing unit 400 may modify the configuration of the multifunctional medical device according to the received commands. For example, the processing unit 400 may change the configuration of the user interface according to the received commands. As another example, the user interface may be changed from displaying a heart rate in numbers to displaying a waveform based upon received ECG signals. In some embodiments, the processing unit 400 is capable of downloading software upgrade packages from a network via the communication module 460.

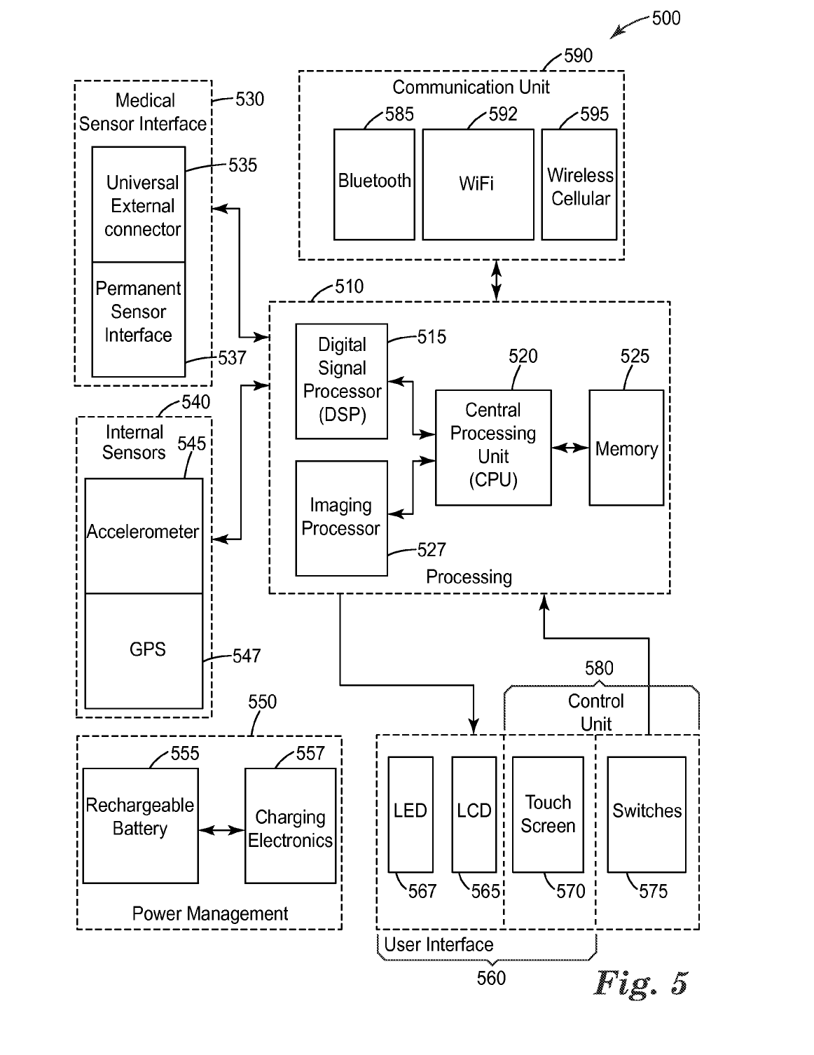

FIG. 5 is a system diagram of an exemplary multifunctional medical device 500 illustrating exemplary components for device 100. The multifunctional medical device includes a medical sensor interface 530 to receive data from medical sensors and send commands to medical sensors. The medical sensor interface 530 may comprise a universal external connector 535, which allows connection of medical sensors which are not hosted within the body of the multifunction medical device 500. The medical sensor interface 530 may also include a permanent internal sensor interface 537 that allows connection of a permanent sensor at least partially within the housing of the device 500. The multifunctional medical device 500 includes a processing unit 510 that receives medical measurement data transmitted via the medical sensor interface 530 and sends command to one or more sensors connected via the universal external connector 535 or the permanent internal sensor interface 537. The processing unit may include a central processing unit (CPU) 520 to compose data for transmission and memory 525 to store data. In some cases, the processing unit may further include a digital signal processor (DSP) 515 to perform signal processing on the received medical measurement signals, and/or an imaging processor 527 to generate display contents.

The multifunctional medical device 500 includes a power management unit 550 to provide power supply. The power management unit may include a rechargeable battery 555 and charging electronics 557. In some cases, the charging electronics may use the universal external connector 535 to connect with a power supply. In some other cases, the charging electronics may use a separate connector (not shown in the diagram) to connect with a power supply.

The multifunctional medical device 500 may further include a communication unit 590 to transmit and receive data. The communication unit 590 may include electronics to provide one or more of short-range wireless communication interfaces, such as interfaces conforming to a known communications standard, such as a Bluetooth standard, IEEE 802 standards (e.g., IEEE 802.11), a ZigBee or similar specification, such as those based on the IEEE 802.15.4 standard, or other public or proprietary wireless protocol. The communication unit 590 may also include electronics to provide one or more of long-range wireless communication interfaces, such as cellular network interfaces, satellite communication interfaces, etc. If a medical sensor is connected to a universal external connector and the medical sensor is identified by the central processing unit 520 as not currently supported by the medical device 500, the central processing unit 520 may request a download of driver through the communication unit 590. In some cases, the device 500 may receive commands from the communication unit 590 and modify the configuration of the device 500 or send commands to the medical sensors in use.

The multifunctional medical device 500 may optionally include a user interface 560. In some cases, the user interface 560 may include one or more LEDs, such as LED 567, or other types of indicators to indicate the status of the device 500 or the configuration of the device 500. For example, when the battery 555 is low in power capacity, a LED may show yellow blinking signal. In some other cases, the user interface 560 may include a LCD display 565, a touch screen 570, or other type of display, to indicate the status of the device 500, the configuration of the device 500, or present data received and/or processed by the processing unit 510 in number or waveform format. In such configuration, an imaging processor 527 may compose the display content to display on the user interface 560.

In some cases, the multifunctional medical device 500 may include a control unit 580. In some cases, the control unit 580 may include buttons or switches 575 to adjust the configuration of the device 500. In some other cases, the control unit 580 may include a touch screen 570 to accept user input/commands. Optionally, the multifunctional medical device 500 may include internal sensors 540 disposed within its housing. The internal sensors 540 may include, as an example, an accelerometer 545 and/or a GPS unit 547. The signals gathered by the internal sensors 540 may be transmitted to the digital signal processor 515 to enhance the signal quality or extract desired data from the signals. The processed data may be used by the central processing unit 520 to change the configuration of the device 500 or transmit via the communication unit 590 to provide information regarding the functioning condition of the device 500. For example, the functioning condition may be the location of the device determined based on signals generated by the GPS unit 547.

When a sensor is initially connected with a multifunctional medical device, the device needs to determine the sensor type and/or the sensor ID, referred to as sensor discovery process. FIG. 6 illustrates an exemplary sensor discovery process for a multifunctional medical device. First, a medical-sensor identification is read (step 610). Here, the medical-sensor identification may uniquely identify the type of the medical sensor, or the medical sensor itself. In some cases, when a medical sensor is connected with the connector, the medical-sensor identification may be first sent to the connector and read by the processing unit. In some cases, the medical-sensor identification may be read by one of the components of the control unit. For example, a RFID tag may be attached to the medical sensor and read by the RFID reader of the control unit. In some other cases, the medical-sensor identification may be read via to a dedicated pin of the connector. After the medical-sensor identification is read, the processing unit may determine the type of the medical sensor according to the identification (step 620). Next, the processing unit may modify the configuration of the multifunctional device according to the medical-sensor type information (step 630). In some cases, the processing unit may download a driver for the medical sensor if necessary.

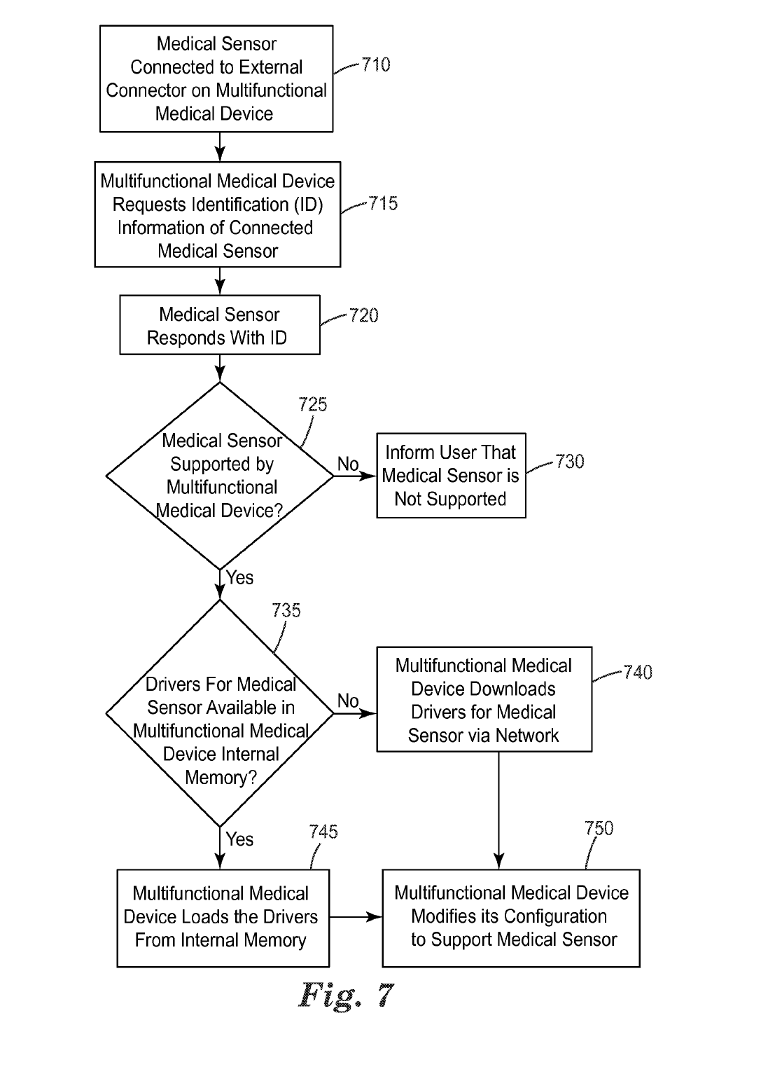

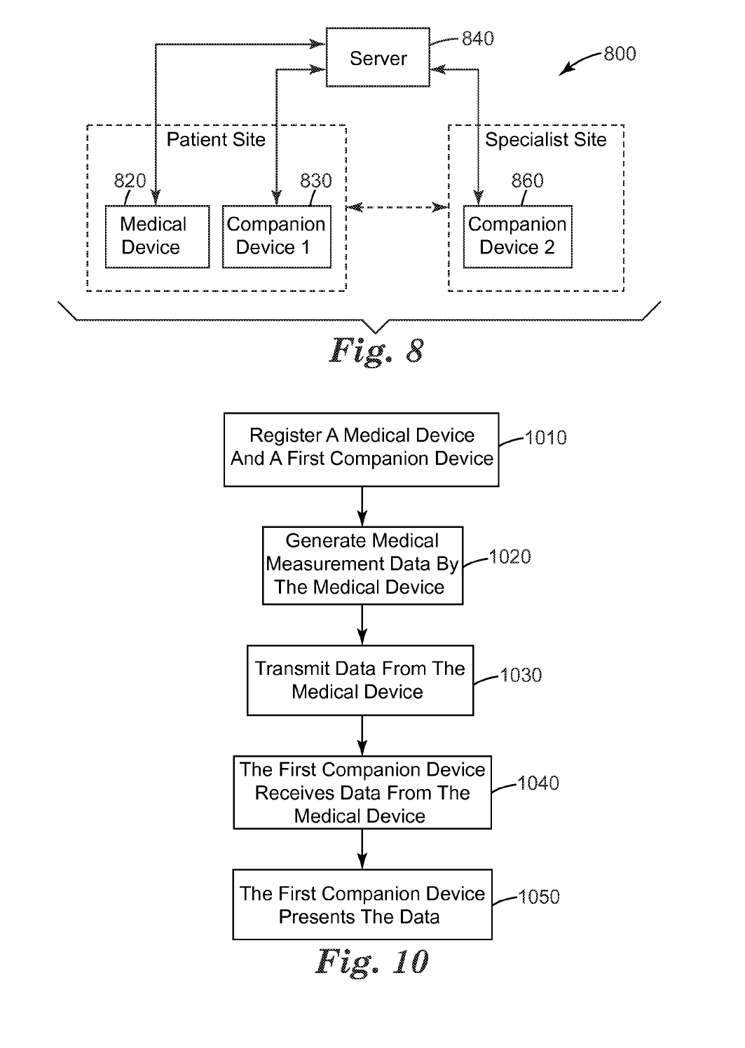

FIG. 7 is another exemplary sensor discovery process of a multifunctional medical device. First, a medical sensor is connected to an external connector on a multifunctional medical device (step 710). Second, the multifunctional medical device requests identification information of the connected medical sensor (step 715). Next, the medical sensor responds by transmitting its identification information (step 720). Based on the medical sensor's identification information, the multifunctional medical device determines whether the medical sensor is supported (step 725). If the sensor is not supported, the device may inform the user that the sensor is not supported (step 730), by changing a status indicator, for example. Next, if the device is supported, the device determines whether the driver for medical sensor is available in the device's memory (step 735). If the driver is not available, the device downloads a driver for the medical sensor via a connected network (step 740). If the driver is available, the device loads the driver from its internal memory to support the medical sensor (step 745). Further, the medical device modifies its configuration to support the sensor (step 750). For example, the display configuration may be modified according the type of medical sensor connected.

Telemedicine Companion System

FIG. 8 is a system diagram of an embodiment of a telemedicine companion system 800. At the patient site, a medical device 820 and a local companion device 830 may be included in the telemedicine companion system 800. The medical device 820 may be a hand-held medical device with wireless capability. In some cases, the medical device 820 may be a multifunctional medical device described in the present disclosure. In some other cases, the medical device 820 may be a modular stethoscope described in the Patent Publication No. 2010-0056956, entitled “Modular Electronic Biosensor with Interface for Receiving Disparate Modules”, which is incorporated herein by reference in its entirety.

A companion device may be a wireless-enabled device with a user interface. For example, a companion device may be a smart-phone, a tablet personal computer (PC), a laptop, a computer, or a personal digital assistant (PDA). The local companion device 830 may receive medical measurement data generated by the medical device 820 and present an indication of the medical measurement data on its user interface. In some cases, the local companion device 830 may accept user commands via its user interface and send the commands to the medical device 820. In some embodiments, the local companion device 830 may perform signal processing and analysis to the received medical measurement data and transmit the analysis result to the specialist site. For example, an exam camera captures a skin image of size 5 cm×5 cm, while a portion of the image of 1 cm×1 cm is of interest, the local companion device 830 may crop the image to the portion of interest manually or automatically before transmitting the image. In some embodiments, more than one medical device used simultaneously at the patient site is linked to the local companion device. In a preferred embodiment, the local companion device 830 may store received medical measurement data and/or the data being processed and/or the analysis result in its internal memory. If a wireless connection is temporarily lost, the local companion device 830 may label the stored data in its internal memory, with time stamp or other sequence indicators, for example. When the wireless connection is later established, the local companion device 830 may transmit the labeled data in its internal memory via the wireless connection.

In some cases, the local companion device may load software designed to support the specific functionality of the medical device 820 when the medical device 820 is active. In some other cases, if the medical device 820 may use more than one type of sensor, the local companion device may load software or change the configuration of the software to support (i.e. process data acquired by the sensor, presents the data, etc.) the active sensor(s) being used by the medical device 820. In some cases, the local companion device 830 may modify the configuration of the user interface based upon the device information received from the medical device 820. In some cases, the local companion device 830 may automatically download device-specific software if the software is not installed on the local companion device 830.

In some embodiments, the telemedicine companion system may include a remote companion device 860 at the specialist site. The remote companion device 860 may receive medical measurement data transmitted by the medical device 820 or the local companion device and present an indication of the medical measurement data to a medical specialist. After reviewing the medical measurement signals, a medical specialist may provide instruction to the patient site via the remote companion device 860. In some embodiments, the telemedicine companion system 800 includes the medical device 820 and the remote companion device 860, but the system 800 does not include the local companion device 830. In some cases, the medical specialist may control the medical device 820 used at the patient site via entering commands using the remote companion device 860. In some other cases, the medical specialist may control the local companion device 830 via entering commands using the remote companion device 860. The commands sent from the remote companion device may be received and interpreted by the local companion device 830 and/or by the medical device 820. For example, the medical specialist may change the filtering mode of the medical device 820, or change the signal processing scheme of the local companion device 830.

In certain embodiments, the telemedicine companion system includes a multifunctional medical device at the specialist site. The control unit of the multifunctional medical device at the specialist site may include a variety of selectable controls and settings for the multifunctional device at the patient site. These settings may be chosen to control the modes, volume, power state, recording settings, and the like of the patient site multifunctional device. In some embodiments, these settings are also selectable via the user interface.

The control unit of the specialist site multifunctional device may also be configured to allow control of some settings on the patient site multifunctional device, while leaving other settings for only local control. For example, the control unit may provide volume control for only the specialist site multifunctional device, while the volume for the patient site multifunctional device is only controllable at the patient site (e.g., via the control unit and/or the user interface on the patient site multifunctional device). In some embodiments, the specialist site multifunctional device may also be configured such that the user interface on the specialist site multifunctional device controls settings of the patient site multifunctional device when connected over a secured network. The specialist site multifunctional device may also be configured to control substantially all of the settings on the patient site multifunctional device (i.e., local control at the patient site is essentially disabled), while still allowing the patient site to control volume.

In some embodiments, the telemedicine companion system 800 does not include the local companion device 830 at the patient site. In such configuration, the medical device 820 at the patient site transmits data to and receives commands from the remote companion device 860 at the specialist site.

In some embodiments, the local companion device 830 or the remote companion device 860 performs an error check on the received data and indicate the data quality on its user interface. If an error is found on the received data, the remote companion device 860 may request the local companion device 830 to transmit the data again.

In some embodiments, either the local companion device 830 or the remote companion device 860 may perform automatic diagnosis to the medical measurement signals. The automatic diagnosis may be, for example, a diagnosis method described in U.S. Pat. No. 7,300,405, entitled “Analysis of Auscultatory Sounds Using Single Value Decomposition,” or described in U.S. Pat. No. 6,572,560 “Multi-modal cardiac diagnostic decision support system and method,” which are incorporated herein by reference in its entirety.

In some embodiments, the telemedicine companion system 800 may include a server 840. The server may be, for example, a computer, a series of computers, a cloud-computing server, and the like. The server 840 may provide device registration and authorization, device administration, and data storage. In some cases, the server may receive registration request from the medical device 820 and the local companion device 830. In some cases, the server may receive medical measurement data from either the medical device 820 or the local companion device 830, store the received data in a data repository, and transmit the received data to the remote companion device 860. In some cases, the server may authenticate the medical device 820 and the local companion device 830 by an authentication scheme, exemplary embodiments of which are described in further detail below. In such configurations, the server may transmit data to the remote companion device 860 only if the medical device 820 and/or the local companion device 830 are authenticated. In other embodiments, transmission may be allowed only if the remote companion device 860 is authenticated in addition to the medical device 820 and/or the local companion device 830. In some cases, the server 840 may exchange IP addresses to each of the registered devices requesting connection, and permit the devices to directly communicate and exchange data via the network.

In a preferred embodiment, the telemedicine companion system uses cellular networks as communication medium. In some embodiments, the remote companion device 860 and the server 840 are within a network security firewall of a healthcare information management system, while the medical device 820 and the local companion device are outside the firewall. Use of a cellular network allows secured communications across the firewall.

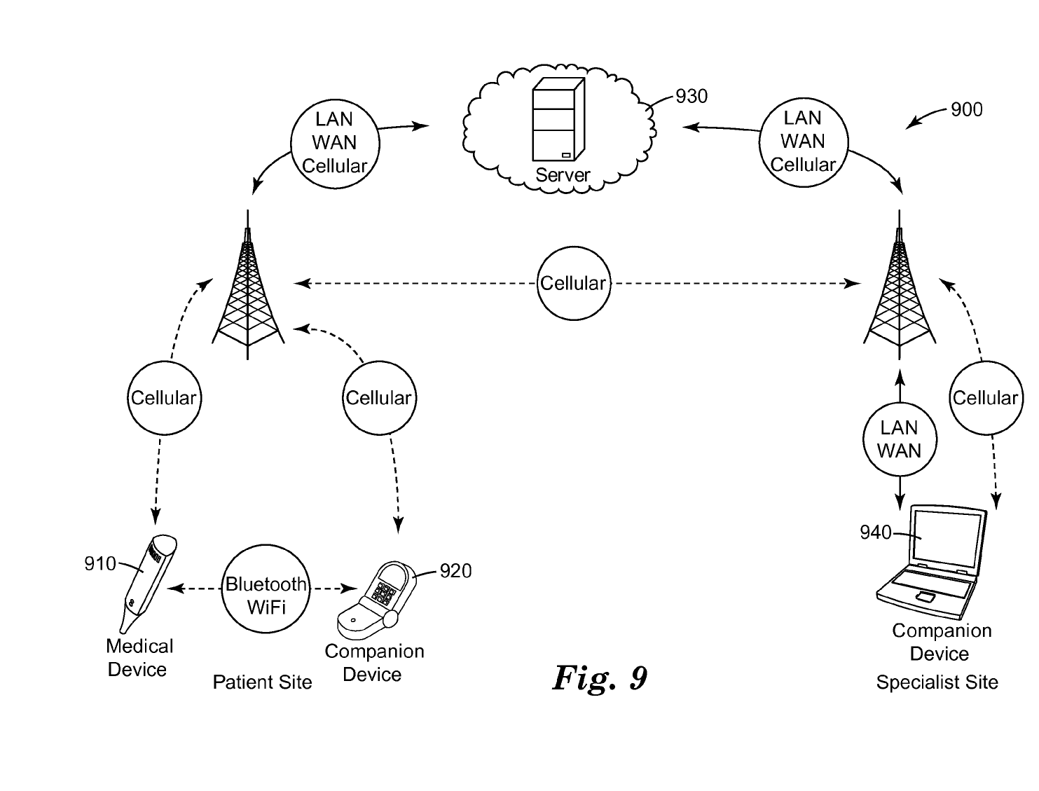

FIG. 9 illustrates another exemplary telemedicine companion system 900. In an exemplary embodiment, a medical device 910 and a local companion device 920 at the patient site may be connected via a cellular network or cellular networks of more than one cellular service providers. A server 930 may be connected to a cellular network. The server 930 may also connected to other wired and/or wireless networks. Further, a remote companion device 940 at the specialist site may be connected to the telemedicine companion system 800 via a wired or wireless network. In some cases, the remote companion device 940 may be connected via a cellular network. In some embodiments, if the medical device 910 and the local companion device 920 are within coverage of a short-range wireless network, such as Bluetooth, or an IEEE 802.11-compliant network, the medical device 910 and the local companion device 920 may establish connection via the short-range wireless network.

FIG. 10 is an exemplary functional flowchart of a telemedicine companion system. First, a medical device and a first companion device are registered on a server (step 1010). The medical device and the first companion device may further be authenticated by the server before the following steps. Next, the medical device generates medical measurement data at the patient site (step 1020). The medical device transmits the data to the first companion device (step 1030). In some cases, the server may receive the data from the medical device and the server may forward the data to the first companion device. The first companion device receives the data (step 1040) and presents an indication of the data on its user interface (step 1050).

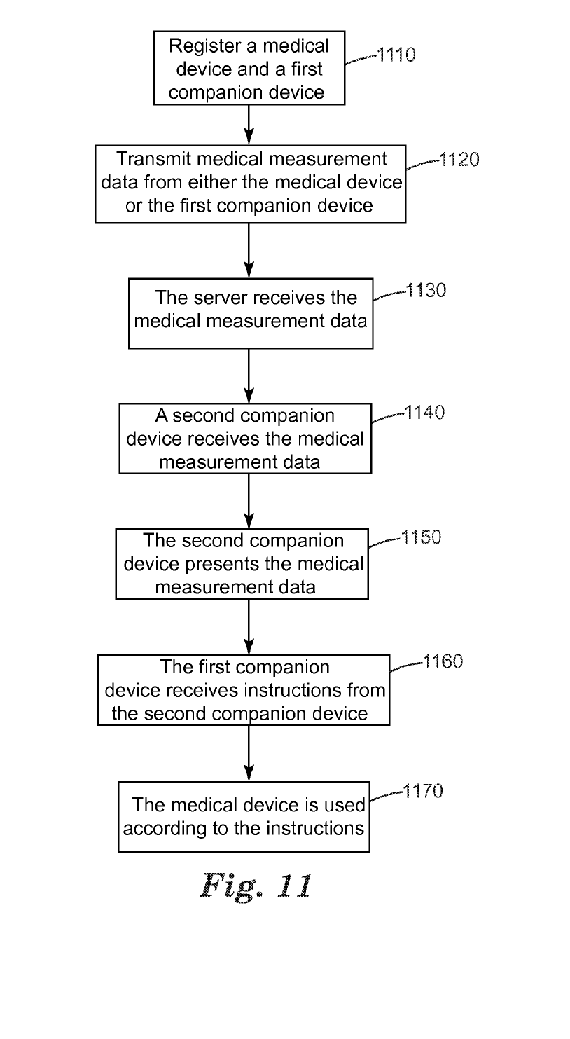

FIG. 11 is another exemplary functional flowchart of a telemedicine companion system. First, as an optional step, a medical device and a first companion device are registered on a server (step 1110). The medical device and the first companion device may further be authenticated by the server before the following steps. Next, medical measurement data is transmitted from either the medical device or the first companion device (step 1120). In some cases, the first companion device may transform the medical measurement signals generated by the medical device to medical measurement data with desirable format and quality, which could be configured by user input via the first companion device or a second companion device. The server receives the medical measurement data (step 1130) and forwards the data to the second companion device (step 1140). Before the server forwards the data to the second companion device, the server may require the second companion device to be properly registered and authenticated. In some cases, the second companion device may receive the medical measurement data transmitted from either the medical device or the first companion device. In some cases, the server stores the medical measurement data at a data repository. Then, the second companion device presents an indication of the medical measurement data on its user interface (step 1150). Optionally, the medical specialist may provide instruction to the patient site via the second companion device (step 1160). Then, the medical device is used according to the instructions (step 1170). For example, the medical specialist may instruct the care giver to move the medical device to certain direction.

In some cases, a medical specialist may provide commands via a second companion device at the specialist site to a first companion device at the patient site. The first companion device may change the setting of the device driver or change the setting of the medical device according to the command. In some other cases, the second companion device may automatically transmit commands to the first companion device after analyzing the data. For example, the second companion device may determine that a data packet is missing and request the first companion device resend the data packet. After receiving the commands, the first companion device operates according to the commands. In certain embodiments, a second multifunctional medical device at the specialist site transmits command signals to the first multifunctional medical device at the patient site.

For telemedicine applications, device registration and authorization can be important for establishing secure data transaction, for example, to maintain patient privacy. In some embodiments, after a medical device or a companion device powers on, the medical device or the companion device may establish a connection to the server. Upon establishment of the connection, the medical device or the companion device may provide the server with a unique identification and authentication information. The authentication information may be, for example, security phrase (password), security certificate, or the like. In some cases, a token-based authentication scheme may be used, where a hardware-based or software based token is required along with authentication information. In some cases, the server may maintain a secure directory list that includes authorized device's identification, authentication information, and other information. The server may authenticate the medical device or the companion device based on the secure directory list. In some other cases, the secure directory list may include pairing information of one or more medical devices with one or more companion devices. In some embodiments, only authenticated devices may make subsequent connections to other devices in the telemedicine system. In some other embodiments, when a remote companion device at the specialist site is used outside the firewall, the remote companion device is required to be authenticated by the server before it sets up connection with a local companion device at the patient site. For example, the remote companion device may be used by a medical specialist when the specialist travels to a rural clinic to provide on-site medical procedure.

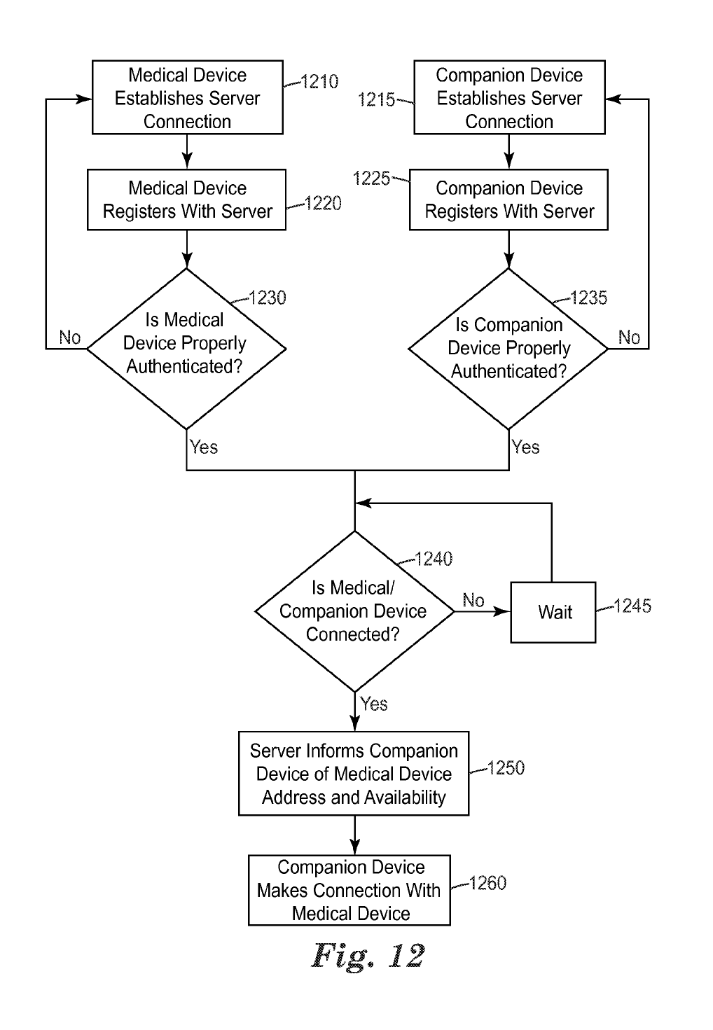

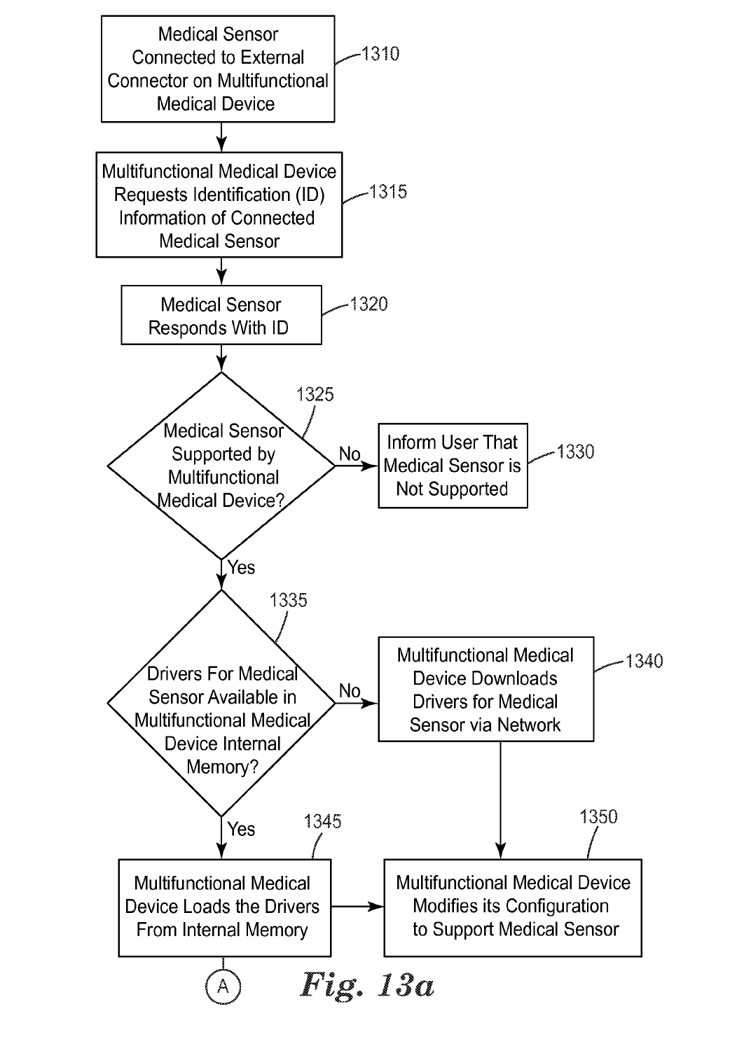

FIG. 12 is a flowchart of an exemplary device registration procedure of a telemedicine companion system. In the beginning, a medical device establishes a connection with the server (step 1210). Next, the medical device may register with the server (step 1220). In some cases, the medical device may provide its identification information to the server during the registration. The server will determine whether the medical device is properly authenticated (step 1230). For example, the server may authenticate the medical device based on the secure directory list. If the medical device is authenticated properly, the medical device will wait for a connection of a companion device (step 1240, step 1245).

On the other side, a companion device establishes a connection with the server (step 1215). Then, the companion device may register with the server (step 1225). The companion device may provide its identification information to the server during the registration. The server will determine whether the companion device is properly authenticated (step 1235). For example, the server may authenticate the companion device based on the secure directory list. If the companion device is authenticated properly, the companion device will wait for a connection of the medical device (step 1240, step 1245). If both the medical device and the companion device are connected with the server, the server may inform the companion device of address and availability of the medical device (step 1250). Last, the companion device may make connection with the medical device (step 1260).

In an alternative embodiment, the server may inform the medical device of address and availability of the companion device, then the medical device may make connection with the companion device. In some cases, the companion device and the medical device may directly communicate each other with the address supplied by the server. In some other cases, the companion device may send data to the server and the server may forward the received data to the medical device; and the medical device may send data to the server and the server may forward the received data to the companion device.

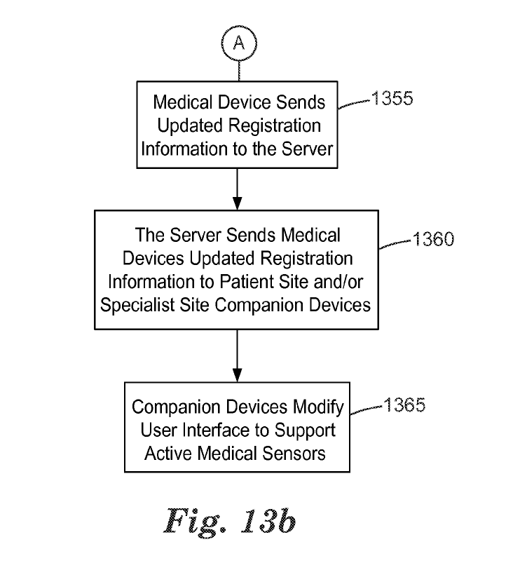

A sensor discovery process to determine the type of active medical sensor for the medical device may be used when a multifunction medical device is utilized in a telemedicine companion system. An exemplary flowchart of a sensor discovery process for a telemedicine companion system is illustrated in FIGS. 13a and 13b. First, a medical sensor is connected to an external connector on a multifunctional medical device (step 1310). Second, the multifunctional medical device requests identification information of the connected medical sensor (step 1315). Next, the medical sensor responds with its identification information (step 1320). Based on the medical sensor's identification information, the multifunctional medical device determines whether the medical sensor is supported (step 1325). If the sensor is not supported, the device may inform the user that the sensor is not supported (step 1330), by changing a status indicator, for example. Next, if the device is supported, the device determines whether the driver for medical sensor is available in the device's memory (step 1335). If the driver is not available, the device downloads a driver for the medical sensor via a connected network (step 1340). If the driver is available, the device loads the driver from its internal memory to support the medical sensor (step 1345). Further, the medical device modifies its configuration to support the sensor (step 1350). For example, the display configuration or control elements may be modified based on the type of the active sensor. After the sensor type is determined, the medical device sends updated registration information to the server (step 1355). The server sends the medical device's updated registration information to companion devices at the patient site and/or the specialist site (step 1360). The companion devices modify their user interface respectively to support the medical sensor in use (step 1365).

For telemedicine applications, a faithful reproduction of medical measurement signals is desirable during data transmissions. Here, faithful reproduction means a digitally exact replica or a sufficiently exact reproduction (with error correction, for example). For streaming data such as ECG or heart sounds, as an example, the digital data is required to exactly replicate the digitized value and timing. When the components of a telemedicine companion system are linked over a secure network connection, signals may be sent between a medical device and a companion device in substantial real-time. In some embodiments, signals may be sent between a medical device and a companion device at the patient site and a server, a remote companion device at the specialist site, in substantial real-time. For example, body sounds captured by the medical device may be transmitted from the local companion device at the patient site to the remote companion device at the specialist site in substantial real-time. The body sounds may also be reproduced at the server in substantial real-time. In addition, medical measurement data may be recorded and stored by either the medical device or the local companion device or both and later transmitted.

In some embodiments, the signals transmitted by the medical device or the local companion device at the patient site and over the network are packetized and enumerated by the medical device or the local companion device, and undergo an error check by either the server or the remote companion device at the specialist site to assure faithful data quality and reliable reproduction at the second companion device at the specialist site. The error check may be performed by each component of the telemedicine companion system (i.e., patient site medical device and local companion device, the optional server, and the remote companion device at the specialist site) as a further assurance of accurate transmission of data from the patient site to the specialist site. The error check may use any suitable data transmission check techniques, including, but not limited to, cyclic redundancy check (CRC), checksum, horizontal and vertical redundancy check, hash function, repetition code, and the like. The system may also incorporate, for example, sample throughput measurements, performed to determine excess data or data starvation in the communication link. In short, the data packets from either the medical device or the local companion device at the patient site are directly relayed (i.e., mirrored) over the network to the remote companion device at the specialist site.

In preferred embodiments, the telemedicine companion system includes an error check and validation independent of the underlying communication system or network (e.g., Bluetooth, Transmission Control Protocol/Internet Protocol (TCP/IP)) protocol. The independent error check may be performed at any component of the telemedicine companion system as a further assurance that the signal is a faithful reproduction of the medical measurement data transmitted from the patient site. In certain preferred embodiments, interruptions in service of the underlying system are classified according to duration and severity, with all errors resulting in a communication (via one or more components of the telemedicine companion system) that the signal is not a faithful reproduction. For example, a patient site component may send a data packet or other signal to a specialist site system component every other 500 milliseconds. An interruption in the underlying communication system or network exceeding 500 milliseconds may result in a dropped packet/signal and a resultant indication at the specialist site of degraded data quality (e.g., via changing color of an indicator).

In some cases, the user interface of the companion device may include one or more of a display component, an audio component, a vibration component, and other components. In some cases, the display component may present the patient information, the medical device information, and an indication of the medical measurement data received. In some cases, the display component is capable of simultaneously presenting data generated by more than one medical device or medical sensor being used at the patient site. In some other cases, the audio component may play back acoustic signals gathered at the patient site. For example, the audio component is a speaker that plays back body sounds recorded by a stethoscope at the patient site. Additionally, the user interface may include a vibration component that vibrates in certain configurable conditions. For example, the vibration component may vibrate when the oxygen saturation measured by a pulse oximeter is lower than 90.

In some embodiments, the user interface of the second companion device and/or the first companion device may further include a visual and/or audio indication of whether the medical measurement data received at the specialist site is a faithful reproduction of the medical measurement data transmitted from the patient site. In some embodiments, the user interface may include a fidelity indicator that changes color to indicate whether faithful reproduction is occurring at the specialist site. For example, the fidelity indicator may be displayed in green when the medical measurement data is a faithful reproduction and in red when the medical measurement data is not a faithful reproduction. This indication may be based on the error detection performed by a server and/or a remote companion device at the specialist site. The error detection may also be performed by the medical device or a local companion device at the patient site. For example, if errors are identified by the server, the server may send a signal to the remote companion device that the data received is not a faithful reproduction as the data sent by the medical device or the local companion device at the patient site. The indication may then be provided by the user interface of the remote companion device, which shows a clinician at the specialist site that the data presented via the remote companion device is not a faithful reproduction. The user interface of the local companion device may also provide an indication of faithful reproduction. In alternative embodiments, the lack of error is reported to system components.

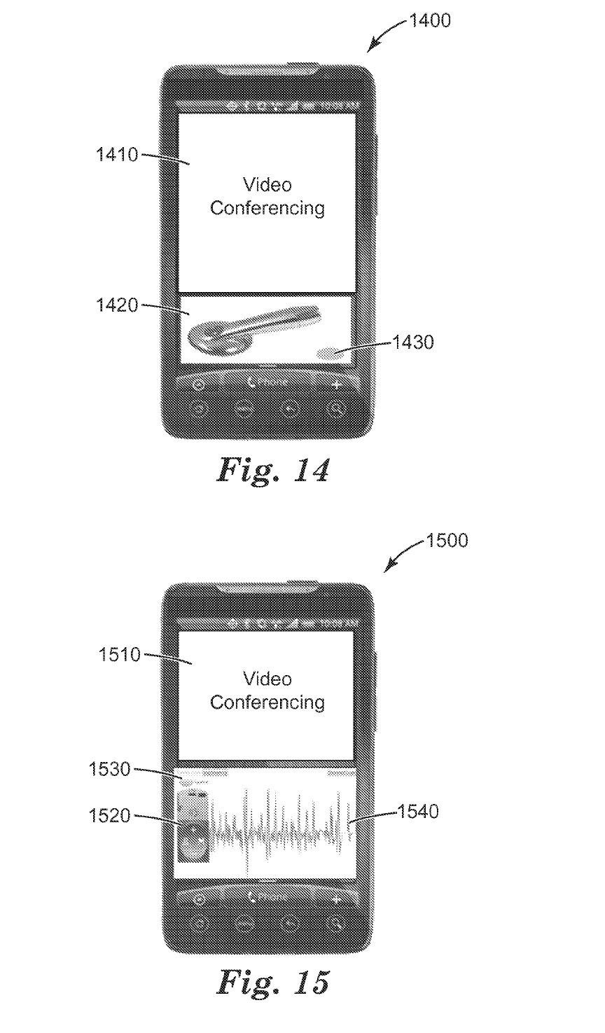

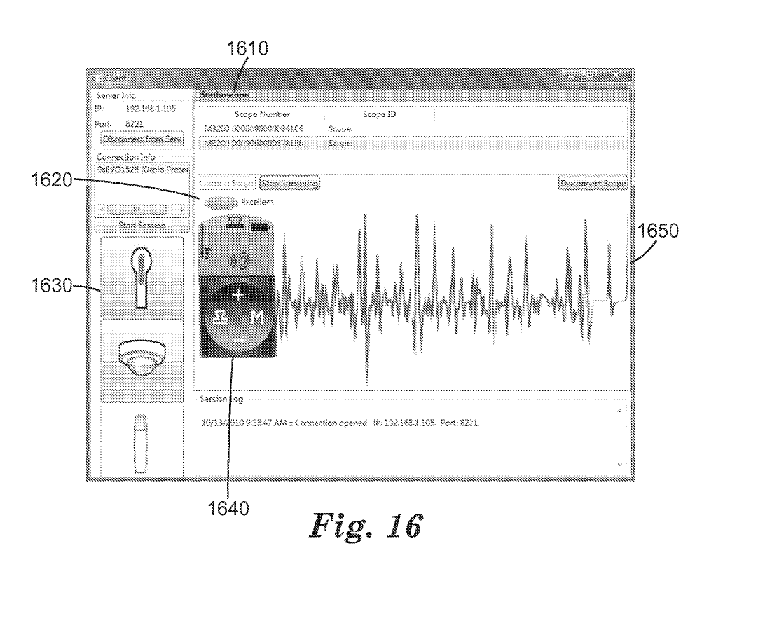

FIG. 14 is a screen shot of an embodiment of a user interface 1400 on a companion device, which may be a hand-held device. The user interface 1400 may include a display section for video conferencing 1410, a display section showing an image of the medical device being used 1420, and a status/mode indicator 1430. FIG. 15 is another screen shot of an embodiment of a user interface 1500 on a companion device. The user interface 1500 may include a display section for video conferencing 1510, a portion of the interface to enter control information to the medical device 1520, status/mode indicator(s) 1530, and a display section for presenting medical measurement data 1540. Another exemplary embodiment of a user interface on a companion device is illustrated in FIG. 16. For example, this may be a user interface on a laptop or a personal computer. The user interface 1600 may include a display section for presenting the medical device information 1610, status/mode indicator(s) 1620, a display section for showing the type of medical sensors in use and allowing user to select one or more sensors 1630, a portion of the interface to enter control information to the medical device 1640, and a display section for presenting medical measurement data 1650.

### EXEMPLARY EMBODIMENTS

1. A multifunctional medical device, comprising:

a housing configured for hand-held manipulation; a processing unit within the housing; a first medical sensor coupled to the housing and in communication with the processing unit, the first medical sensor operable to generate medical measurement signals; and a connector coupled to the processing unit and operable to connect to a plurality of different types of medical sensors, each type of medical sensors operable to generate corresponding medical measurement signals, wherein the processing unit is configured to determine a type of a second medical sensor connected to the connector, receive medical measurement signals from the first medical sensor and the second medical sensor connected to the connector, and transmit the received signals via a network, wherein the first medical sensor and the second medical sensor are capable of operating simultaneously when in use.

2. The multifunctional medical device of embodiment 1, wherein the housing is sealed.
3. The multifunctional medical device of embodiment 1, wherein the processing unit is configured to process the received signals based upon the corresponding type of the medical sensor that generates the received signals, create data packets based upon the processed signals, and transmit the data packets via a network.
4. The multifunctional medical device of embodiment 1, wherein the first medical sensor comprises a transducer that senses auscultation signals, positioned adjacent to the first end of the housing.
5. The multifunctional medical device of embodiment 1, wherein the connector is on the outer surface of the housing and configured to physically connect to a medical sensor.
6. The multifunctional medical device of embodiment 1, wherein the first medical sensor is an exam camera, wherein the housing has a first end, and a second end opposite the first end, and wherein the exam camera is positioned on the first or the second end of the housing.
7. The multifunctional medical device of any one of embodiments 1 to 6, wherein the connector comprises a wireless connection port configured to wirelessly connect to a medical sensor.
8. The multifunctional medical device of any one of embodiments 1 to 6, wherein the first medical sensor and the second medical sensor comprise one or more of a stethoscope, an electrocardiography sensor, a glucose meter, a pulse oximeter, an exam camera (this includes endoscopes & otoscopes per the spec), a sphygmomanometer, a spirometer, a thermometer, an ophthalmoscope,
9. The multifunctional medical device of any one of embodiments 1 to 6, further comprising: a user interface on a portion of the outer surface of the housing, wherein at least a portion of the user interface displays an indication of a type of medical sensor based upon the type of the active medical sensor.
10. The multifunctional medical device of embodiment 9, wherein the housing has an elongated body, wherein the user interface is at least partially viewable in hand-held use, and wherein the user interface displays the medical measure signals from at least one of the first and the second medical sensor.
11. The multifunctional medical device of any one of embodiments 1 to 6, further comprising a control unit for generating control signals, wherein the housing has an elongated body, and wherein the control unit is at least partially accessible in hand-held use.
12. The multifunctional medical device of embodiment 11, wherein the control unit includes at least one of an accelerometer, a gyroscope, an audio transducer, and an ambient light sensor, located in the housing and operably connected to the processing unit.
13. The multifunctional medical device of embodiment 12, further comprising a user interface, wherein the configuration of the user interface is changed based upon the control signals.
14. The multifunctional medical device of embodiment 12, wherein the control unit comprises a GPS unit for determining geographically related information, wherein the processing unit receives the geographically related information from the GPS unit and transmits the geographically related information via a network.
15. An multifunctional medical device, comprising: a housing configured for hand-held manipulation, the housing having an outer surface, an elongated body, a first end, and a second end opposite the first end; a processing unit within the housing; an exam camera coupled to the processing unit, positioned on the first end of the housing; and a connector on the outer surface of the housing, the connector coupled to the processing unit and operable to connect to a plurality of different types of medical sensors, each type of medical sensors operable to generate corresponding medical measurement signals, wherein the processing unit is configured to determine a type of a medical sensor connected to the connector, and receive medical measurement signals from the exam camera and the medical sensor connected to the connector.
16. A system, comprising:

a medical device comprising a housing configured for hand-held manipulation, a communication port for receiving medical measurement signals generated from a medical sensor, a processor within the housing configured to receive the medical measurement signals and transmit a first data indicative of the medical measurement signals via a first secured wireless connection; and a first companion device comprising a user interface, the first companion device configured to receive the first data from the medical device via a second secured wireless connection, and present an indication of the first data on the user interface of the first companion device, wherein the medical device is capable of sending a request to a server to register and authenticate the medical device, and wherein the first companion device is capable of sending a request to a server to register and authenticate the first companion device.

17. The system of embodiment 16, further comprising: a second companion device comprising a user interface, the second companion device configured to receive a second data transmitted from at least one of the medical device and the first companion device, and present an indication of the second data on user interface of the second companion device.
18. The system of embodiment 16 or 17, wherein the communication port comprises a connector on the outer surface of the housing and configured to physically connect to a medical sensor, the connector is operable to connect to a plurality of different types of medical sensors.
19. The system of embodiment 16, wherein the medical device is configured to communicate only with the first companion device that has been authenticated on the server.
20. A method, comprising: generating, by a medical device, medical measurement signals; transmitting, by the medical device, a first data indicative of the medical measurement signals via a first secured wireless network; receiving, by a first companion device, the first data from the medical device via a second secured wireless network; presenting, by the first companion device, an indication of the first data on a user interface; registering and authenticating, by a server, the medical device based upon a request transmitted from the medical device; and registering and authenticating, by the server, the first companion device based upon a request transmitted from the first companion device.
21. The method of embodiment 20, further comprising: receiving, by a second companion device, a second data transmitted from at least one of the medical device and the first companion device; and presenting, by the second companion device, an indication of the second data on a user interface.
22. The method of embodiment 20 or 21, wherein the communication port comprises a connector on the outer surface of the housing and configured to physically connect to a medical sensor, the connector is operable to connect to a plurality of different types of medical sensors.

1. A multifunctional medical device, comprising:
a housing configured for hand-held manipulation;
a processing unit within the housing;
a first medical sensor coupled to the housing and in communication with the processing unit, the first medical sensor operable to generate medical measurement signals; and
a connector coupled to the processing unit and operable to connect to a plurality of different types of medical sensors, each type of medical sensors operable to generate corresponding medical measurement signals,
wherein the processing unit is configured to determine a type of a second medical sensor connected to the connector, receive medical measurement signals from the first medical sensor and the second medical sensor connected to the connector, and transmit the received signals via a network,
wherein the first medical sensor and the second medical sensor are capable of operating simultaneously when in use.

2. The multifunctional medical device of claim 1, wherein the processing unit is configured to process the received signals based upon the corresponding type of the medical sensor that generates the received signals, create data packets based upon the processed signals, and transmit the data packets via a network.

3. The multifunctional medical device of claim 1, wherein the first medical sensor comprises a transducer that senses auscultation signals, positioned adjacent to the first end of the housing.

4. The multifunctional medical device of claim 1, wherein the connector is on the outer surface of the housing and configured to physically connect to a medical sensor.

5. The multifunctional medical device of claim 1, wherein the first medical sensor is an exam camera, wherein the housing has a first end, and a second end opposite the first end, and wherein the exam camera is positioned on the first or the second end of the housing.

6. The multifunctional medical device of any one of claim 1, wherein the first medical sensor and the second medical sensor comprise one or more of a stethoscope, an electrocardiography sensor, a glucose meter, a pulse oximeter, an exam camera, a sphygmomanometer, a spirometer, a thermometer, and an ophthalmoscope.

7. The multifunctional medical device of any one of claim 1, further comprising:
a user interface on a portion of the outer surface of the housing, wherein at least a portion of the user interface displays an indication of a type of medical sensor based upon the type of the active medical sensor.

8. The multifunctional medical device of any one of claim 1, further comprising a control unit for generating control signals, wherein the housing has an elongated body, and wherein the control unit is at least partially accessible in hand-held use, and wherein the control unit includes at least one of an accelerometer, a gyroscope, an audio transducer, and an ambient light sensor, located in the housing and operably connected to the processing unit.

9. The multifunctional medical device of claim 8, further comprising a user interface, wherein the configuration of the user interface is changed based upon the control signals.

10. An multifunctional medical device, comprising:
a housing configured for hand-held manipulation, the housing having an outer surface, an elongated body, a first end, and a second end opposite the first end;
a processing unit within the housing;
an exam camera coupled to the processing unit, positioned on the first end of the housing; and
a connector on the outer surface of the housing, the connector coupled to the processing unit and operable to connect to a plurality of different types of medical sensors, each type of medical sensors operable to generate corresponding medical measurement signals,
wherein the processing unit is configured to determine a type of a medical sensor connected to the connector, and receive medical measurement signals from the exam camera and the medical sensor connected to the connector.

11. A system, comprising:
a medical device comprising
a housing configured for hand-held manipulation,
a communication port for receiving medical measurement signals generated from a medical sensor,
a processor within the housing configured to receive the medical measurement signals and transmit a first data indicative of the medical measurement signals via a first secured wireless connection; and
a first companion device comprising a user interface, the first companion device configured to receive the first data from the medical device via a second secured wireless connection, and present an indication of the first data on the user interface of the first companion device,
wherein the medical device is capable of sending a request to a server to register and authenticate the medical device, and wherein the first companion device is capable of sending a request to a server to register and authenticate the first companion device.

12. The system of claim 11, further comprising:
a second companion device comprising a user interface, the second companion device configured to receive a second data transmitted from at least one of the medical device and the first companion device, and present an indication of the second data on user interface of the second companion device.

13. The system of claim 11, wherein the communication port comprises a connector on the outer surface of the housing and configured to physically connect to a medical sensor, the connector is operable to connect to a plurality of different types of medical sensors.

14. The system of claim 11, wherein the medical device is configured to communicate only with the first companion device that has been authenticated on the server.

15. A method, comprising:
generating, by a medical device, medical measurement signals;
transmitting, by the medical device, a first data indicative of the medical measurement signals via a first secured wireless network;
receiving, by a first companion device, the first data from the medical device via a second secured wireless network;
presenting, by the first companion device, an indication of the first data on a user interface;
registering and authenticating, by a server, the medical device based upon a request transmitted from the medical device; and
registering and authenticating, by the server, the first companion device based upon a request transmitted from the first companion device.
